(19) United States
(12) Patent Application Publication
(10) Pub. No.: US 2008/0243020 A1
(43) Pub. Date: Oct. 2, 2008

(54) Physiological Signal Collecting And Monitoring Device And System

(76) Inventor: Chang-An Chou, Taiwan (CN)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/088,081

(22) PCT Filed: Oct. 8, 2006
(86) PCT No.: PCT/CN2006/002632

§ 371 Date: Mar. 25, 2008

(30) Foreign Application Priority Data
Oct. 8, 2005 (CN) 200510113541.6
Sep. 22, 2006 (CN) 200610138988.3

(51) Int. Cl. A61B 5/08

(52) U.S. Cl. 600/538

(57) ABSTRACT

A physiological signal collecting and monitoring system includes a physiological signal sensing device (10) and a computer device (48). The sensing device (10) includes a physiological signal sensing unit for detecting physiological signals from a subject, a circuit arrangement including a processor for executing a preloaded program to receive the physiological signals from the sensing unit, process thereof and store resultant physiological information into a memory, and a carrier (14) for carrying the sensing unit and the circuit arrangement to attach to the subject. The computer device (48) having a standard computer communication port is used for further processing the physiological information. Furthermore, the sensing device (10) further includes a communication interface to communicate with the standard computer communication port, and the interface to communicate with the processor and memory are integrated into a removable module (16), and the removable module (16) is detachable from the sensing device for acting as a computer dongle so that the removable module (16) is capable of electrically connecting with the computer device (48) to download the physiological information stored in the removable module (16) into the computer device (48).

### FIELD OF THE INVENTION

The present invention is related to a physiological signal collecting and monitoring device and system, and more particularly, to a physiological signal collecting and monitoring device, which can be attached to a user, and a docking unit or computer device corresponding thereto.

### BACKGROUND OF THE INVENTION

Physiological signal monitoring has become more and more important in the modern life since people have paid great attention to their health and physical condition owing to the improved quality of life. Therefore, the development of the devices used for physiological monitoring, no matter in which physiological field, is increasingly growing. For example, researches about sleep quality and sleep disorder have been reported extensively.

The following takes the sleep physiological signal monitoring as the example to explain the development of physiological signal monitoring device.

The traditional physiological signal monitoring and detection related to sleep is held in a sleep laboratory. In the sleep laboratory, various physiological signals, for example, respiration, snoring, blood oxygenation, electrocardiogram, electromyogram, electroencephalogram, and electrooculogram (which are also called polysomngraphy), are exercised. However, for the testee, it is actually an uncomfortable experience to sleep in the unfamiliar sleep environment, where the testee has to connect to numerous connecting wires, which come from bulky machines aside the bed, and once the wires have been disposed, the mobility of the testee is highly restricted. Thus, obviously, these situations do influence the normal sleep conditions of the testee and also the measuring results thereof.

Therefore, for freeing the testee from the numerous wires and also the bulky machines, sleep physiological signal monitoring devices have been developed in various portable types.

At the beginning, like U.S. Pat. No. 5,275,159, a portable device has been disclosed, in which the number of physiological sensors has been decreased for reducing the volume of the device. However, the device still occupies too much space for the patient to carry, as shown in this patent.

Then, more progressive physiological monitoring devices are reported. In one developing aspect, the device is miniaturized for attaching to the human body. For example, the monitoring devices disclosed in U.S. Pat. No. 6,811,538 and U.S. Pat. No. 6,171,258 can be attached to the forehead, and by using this kind of device, sensors disposed out of the forehead are connected thereto through connecting wires.

According to the descriptions above, no matter which kind of monitoring devices, the purpose of simplification and lightweight remains unchanged. Since the purpose is also conformed to the tendency to consider physiological monitoring as a part of homecare system, a user-friendly design with effective cost becomes an important issue.

Therefore, as the devices disclosed in U.S. Pat. No. 6,368,287 and U.S. Pat. No. 6,597,944, for further reducing the size and the complexity, the number of detected physiological parameters is cut down to only one to two, that is, only one to two kinds of signals are used to determine the target physiological situation. This kind of monitoring device may provide the testee an effective method to check and screen the target symptoms. However, on the other hand, the accuracy and completeness in measurement are somehow sacrificed.

Even for devices used to monitor physiological signals during the period other than sleep, a similar development as described above may also be observed. The only difference is that, during the sleep monitoring period, the comfort and convenience are highly demanded. For example, U.S. Pat. No. 6,454,708 provides a monitoring device which might partially solve the problems described above, wherein the device is attached to the chest for collecting data and a smart card is used to drive electrode measurement, receive signals and store data, and after monitoring, the smart card is accessed by a smart card reader. However, the employment of smart card still has some disadvantages, such as, the cost thereof is high, the design thereof is hard to change, and the access to the data is restricted to the smart card reader, so that a physiological monitoring device adopting this design is still hard to spread widely.

### SUMMARY OF THE INVENTION

Therefore, according to the drawbacks described above, the present invention provides a physiological signal collecting and monitoring device and a system corresponding thereto for achieving advantages of compact size, expandable structure, convenient operation, and effective cost.

For achieving the object described above, the present invention provides a physiological signal collecting and monitoring system including a physiological signal sensing device which may work with a computer device or a docking unit.

In a first aspect of the present, the sensing device and the computer device are employed. The sensing device may include a physiological signal sensing unit for detecting physiological signals from a subject, a circuit arrangement and a carrier for carrying the sensing unit and the circuit arrangement to attach to the subject, and further, the computer device, having a standard computer communication port, is used for further processing the physiological information. And, the sensing device may further include a communication interface to communicate with the standard computer communication port.

In a preferred embodiment, the circuit arrangement will include a processor and a memory, in which the processor may execute a preloaded program to receive the physiological signals from the physiological signal sensing unit, process thereof and store resultant physiological information into the memory. Particularly, the communication interface together with the processor and the memory are integrated into a removable module, which is detachable from the sensing device for acting as a computer dongle, so that the removable module is capable of electrically connecting with the computer device to download the physiological information stored in the removable module into the computer device. Preferably, the circuit arrangement may further include a RF module for executing a wireless input and output, in which the RF module can be implemented to wirelessly output an alarm signal when the physiological signals match a preset condition, and it also can be integrated into the removable module.

In another preferred embodiment, the circuit arrangement will include a processor and a RF module, in which the processor may execute a preloaded program to receive the physiological signals from the physiological signal sensing unit, process thereof and wirelessly transmit resultant physiological information via the RF module, and particularly, the communication interface together with the processor and the RF module are integrated into a removable module, which is detachable from the sensing device for acting as a computer dongle, so that the removable module is capable of electrically connected with the computer device for proceeding a system setting of the physiological signal sensing device from the computer device. Preferably, the RF module can be implemented to execute a real time transmission, and/or the RF module can further be implemented to wirelessly output an alarm signal as the physiological signals match a preset condition.

According to the description above, preferably, the sensing unit may be implemented to have at least a sensor/electrode, which is selected from a group consisting of a respiratory sensor, a blood oxygenation sensor, a snoring sensor, an ECG electrode, an EMG electrode, an EOG electrode and an EEG electrode, and besides, the sensing device may further include a sensor/electrode connecting port for connecting with an external sensor/electrode, which can be selected from a group consisting of a respiratory sensor, a blood oxygenation sensor, a snoring sensor, an ECG electrode, an EMG electrode, an EOG electrode and an EEG electrode.

Advantageously, the carrier is made of flexible material, the electrical connection between the removable module and the computer device is achieved by a socket adapter, the standard computer communication port is selected from a group consisting of: USB, 1394, serial port, parallel port and other wired transmission interface, and the removable module can be separated from the physiological signal sensing device by setting a connector therebetween, tearing, cutting or other separating methods.

In a second aspect of the present invention, a sensing device and a docking unit are employed. The physiological signal sensing device, as described above, may include a physiological signal sensing unit for detecting physiological signals from a subject, a circuit arrangement including a processor and a memory, in which the processor may execute a preloaded program to receive the physiological signals from the physiological signal sensing unit, process thereof and store resultant physiological information into the memory, and a carrier for carrying the physiological signal sensing device and the circuit arrangement to attach to the subject. The docking unit may include a displaying element, a power source, and a housing. Particularly, a removable module including the processor and the memory is further defined for being detached from the sensing device and then directly connected to the docking unit, and the docking unit is driven by the processor for displaying the physiological information.

According thereto, the processor is preferably implemented to further analysis the physiological information for obtaining an analysis result, and advantageously, the display element can be selected from a group consisting of: digit display, LCD, LED, and other displaying elements, and the docking unit may further include an operation interface for being used during physiological information access

In a preferred manner, the docking unit may further include a communication module for connecting to an external device in a wireless or wired manner, wherein the wired manner is achieved by one selected from a group consisting of: USB, 1394, UART, SPI, Ethernet and other wired transmission interface, and the wireless manner is achieved by one selected from a group consisting of: Bluetooth, IrDa, 802.11x, and other RF communication protocols.

### BRIEF DESCRIPTION OF THE DRAWINGS

A more detailed understanding of the invention may be had from the following description of a preferred embodiment, given by way of example, and to be understood in conjunction with the accompanying drawings, wherein:

FIG. 1 is a schematic view showing a physiological signal sensing device, which is implemented as an attachable respiratory airflow sensing device, in a preferred embodiment according to the present invention;

FIG. 2 is an implementation diagram showing the respiratory airflow sensing device in FIG. 1 attached on a user;

FIG. 3 is a block diagram showing the circuits of the physiological signal sensing device according to the present invention;

FIGS. 4A and 4B are schematic views showing the deployments of removable module, carrier and electrode/sensor connecting port(s) according to the present invention;

FIG. 5 is a schematic view showing the physiological signal sensing device along with a docking unit in a preferred embodiment according to the present invention;

FIG. 6 is a schematic view showing the physiological signal sensing device along with a computer device in a preferred embodiment according to the present invention;

FIG. 7 is an implementation diagram showing a respiratory airflow sensing device connected with an external snoring sensor, deployed on the nose, and an external SPO2 sensor, located on the ear, in a preferred embodiment according to the present invention; and

FIG. 8 is an implementation diagram showing an ECG device connected with an external snoring sensor, disposed on the throat, and a respiratory effort sensor, positioned on the thorax and the abdomen, in a preferred embodiment according to the present invention.

### DETAILED DESCRIPTION OF THE INVENTION

The present invention is related to an attachable physiological signal sensing device for collecting and monitoring physiological signals in a lightweight and convenient way, and also a system corresponding thereto.

For clarification, a respiration sensing device is taken as an example according to the present invention. However, it should be noticed that the present invention can be applied to all kinds of physiological signal sensing devices without limitation.

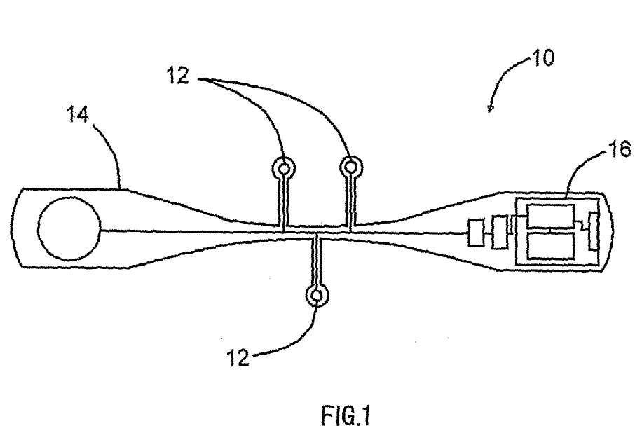

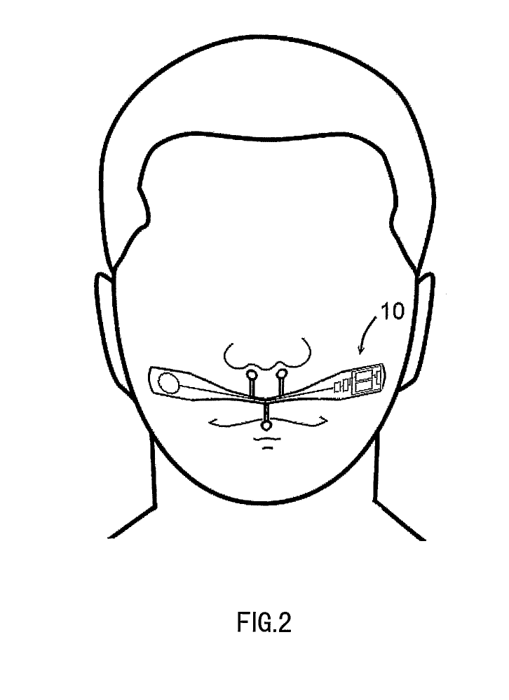

Referring to FIG. 1, the physiological signal sensing device 10 includes a physiological signal sensing unit 12, a circuit arrangement, and a carrier 14, wherein the carrier 14 is used for carrying the physiological signal sensing unit 12 and the circuit arrangement to attach to a measuring position on the user, and as shown in FIG. 1, the circuit arrangement is implemented to distribute over the carrier and the circuits thereof are not located in a specific area. Here, depending on the physiological signals to be detected, the carrier 14 can be attached to different positions. For example, but not limited, if a respiration detection is executed, the carrier might be attached to a position between the nose and the mouth, or if an electromyography detection is executed, the carrier might be attached to the cheeks (for monitoring tooth grinding) or limbs (for monitoring PLMS (periodic leg movement syndrome) or RLS (restless legs syndrome)), or if an electrocardiography detection is executed, the carrier might be attached to a position close to the heart. Furthermore, since the carrier 14 is used to attach to the body surface, to fit the curvy surface of human body, the carrier 14 is preferably made by a flexible material, such as a flexible PCB or any flexible material capable of carrying the sensing unit and circuit arrangement. In addition, the surface of the carrier 14 for contacting the user's skin is preferably implemented to have stickiness, such as by applying an adhesive, to prevent the sensing device 10 falling from the skin. For example, when executing the respiration detection, as shown in FIG. 2, the user only needs to aim the sensing unit at the nostrils and mouth, attach the carrier 14 to the cheeks, then the arrangement of the sensing device 10 before monitoring is accomplished.

Except for directly applying an adhesive on the surface of the carrier, it also can be implemented to use an additional attaching element, such as adhesive tape, for attaching the device to body surface. That is, the attaching method of the carrier is not limited.

According to the present invention, the physiological signal sensing unit 12 of the sensing device 10 is used to collect signals from the attached position, wherein the sensor/electrode used by the sensing unit 12 is the conventional physiological signal sensor, for example, airflow sensor or heat sensor for respiration detection between nostrils and mouse, movement sensor or EMG electrode for limb movement detection, piezoelectric component or microphone for snoring detection, ECG electrode for ECG detection, etc. Here, it should be noticed that the quantity employed in the sensing unit 12 is not limited.

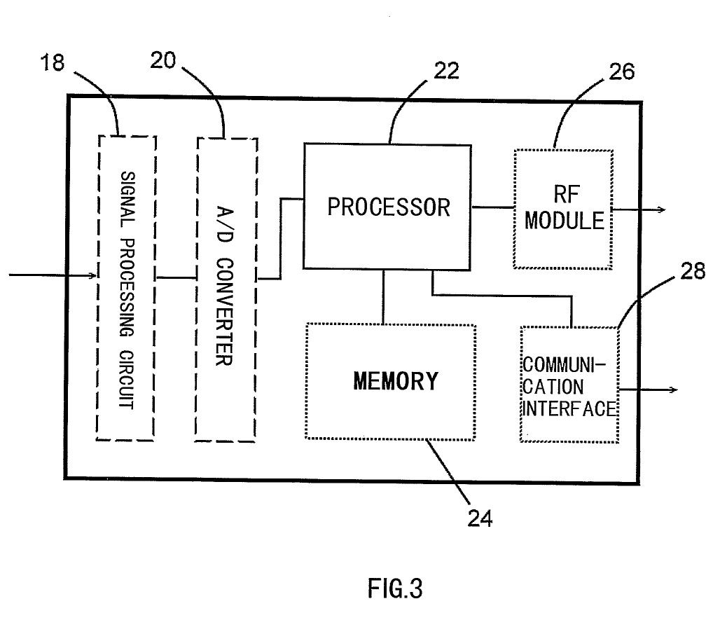

As to the circuit arrangement, it represents all the circuits employed by the present invention for processing the physiological signals, for example, FIG. 3 depicts one possibility of the circuit arrangement. The circuit arrangement, for example, may includes, but not limited, a signal processing circuit 18 (such as filtering and amplification circuits) for primarily processing the collected physiological signals, an A/D (analog-to-digital) converter 20 for converting the physiological signal into a digital form, a processor 22 for processing and analyzing digital signals so as to obtain a physiological information, a memory 24 for storing the physiological information, such as flash memory or EEPROM, a RF module 26 for wireless transmission, and a power source for supplying the power to the sensing device, such as battery. All these circuits are only described for illumination and not for restriction, and circuits which can be used to process physiological signals may all belong to the range of the present invention.

Furthermore, according to the concept of the present invention, in the physiological system, the sensing device may work together with a docking unit or a computer device, wherein the sensing device 10 and the docking unit/computer device are combined through a removable module 16 (as shown in FIG. 1), which is further defined from the circuit arrangement of the sensing device 10.

One of the advantages to make the module 16 removable is cost-saving. Thereby, when the carrier 14 is implemented to be disposable, the reusable circuits can be integrated into the removable module. Therefore, the quantity and type of circuits included in the removable module 16 are not limited, for example, FIG. 1 shows one possibility thereof in which only parts of the circuits are classified as the removable module. In FIG. 3, different types of lines are used to express different categories of circuits, wherein dashed lines represent the circuits which might not be included in the removable module, the dotted lines represent the circuits which are not necessarily present in the circuit arrangement but might be included in the removable module if present, and the solid lines represent the circuits which must be included in the circuit arrangement and also in the removable module.

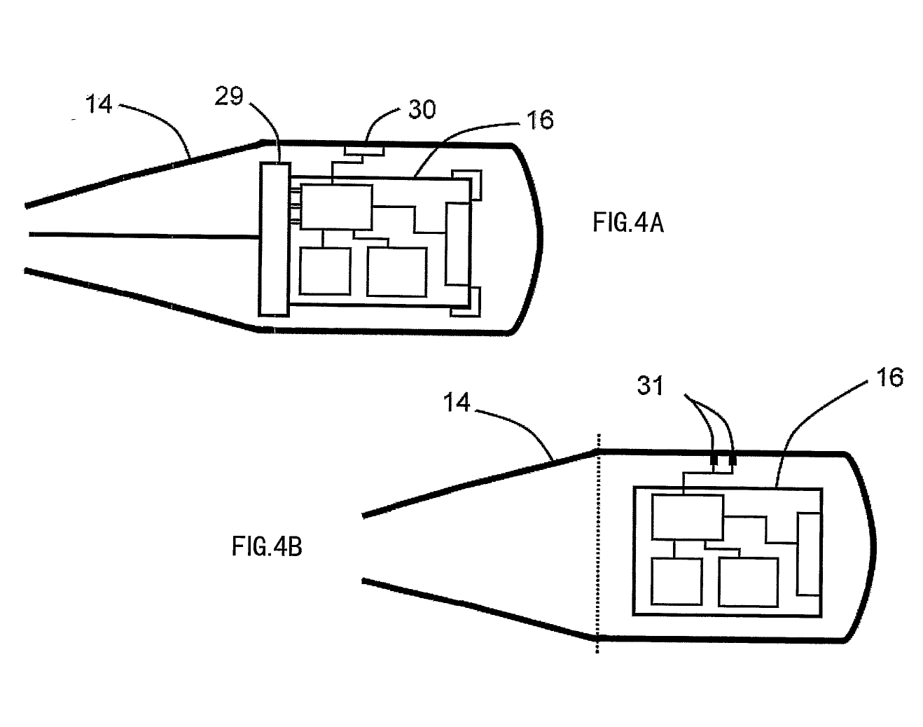

Since the removable module 16 can be detached from the carrier 14, as shown in FIG. 4, a mechanism for achieving the separation therebetween must exist. There are various types of mechanism, for example, as shown in FIG. 4A, the removable module 16 can be mounted on and removed from the carrier 14 through a connector 29 (no matter whether the carrier 14 is disposable or not), or, as shown in FIG. 4B, a direct destruction, such as cutting or tearing, can be adopted if the cost of circuits is acceptable and the re-use becomes unnecessary. And after destruction, the removable module 16 might be removed with a part of the carrier 14 and the rest circuits (which might be different according to the different circuits included in the removable module) are left on the remainder part of the carrier 14.

Furthermore, the following examples explain the relationship between the removable module 16 and the docking unit/computer device, wherein the removable module 16 may accordingly have different improvements on convenience, cost, or universality.

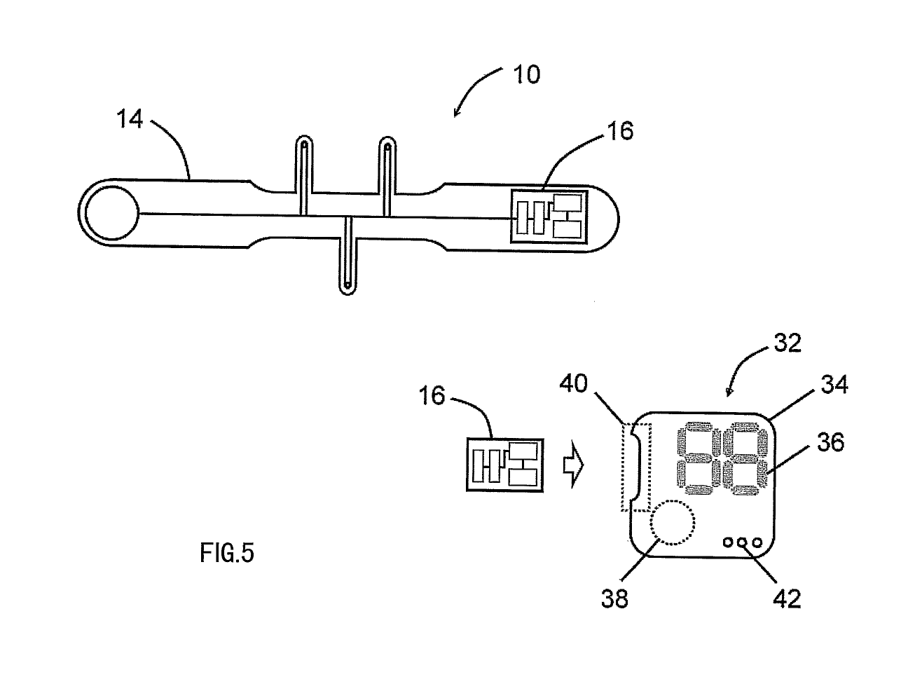

In one embodiment of the present invention, FIG. 5 shows the sensing device 10 and the docking unit 32 which can be directly connected with the removable module 16, wherein the docking unit 32 includes a housing 34, a displaying element 36 and a power source 38, and the removable module 16 at least includes, but not limited, the processor 22 and the memory 24.

In this embodiment, the removable module 16 and the docking unit 32 are combined by direct plugging, as shown in FIG. 5, so that the docking unit 32 may further includes a socket 40 which opening matches to the shape of the removable module 16.

Moreover, after removing from the carrier 14 and plugging into the docking unit 32, it is particular that the removable module 16 is further employed to drive the docking unit 32, that is, the docking unit would not be operated until the removable module 16 is plugged in. Therefore, after the combination, the displaying element 36 may display the physiological information stored in the memory 24, and preferably, the processor 22 may further execute a preloaded program so as to perform additional functions and display more information.

In other words, the processor 22 which originally belongs to the physiological signal sensing device 10 is shared by both the removable module 16 and the docking unit 32 so that not only the cost efficiency can be improved, but a basic demand on viewing the result also can be satisfied. Therefore, this design is quite suitable for the user who is incapable of operating the computer or who does not own a computer. Besides, the additional advantage is that since the removable module 16 is combined with the docking unit 32 after totally separated from the sensing device 10, the removable module 16 can not be accessed as the sensing device 10 still remains on the body surface of the user so that it provides a solution to the issue of power isolation.

Furthermore, the docking unit 32 may further include an operation interface 42 for regular operations, such as power on, power off, menu selection, etc. For example, if the preloaded program executed by the processor provides more complicated and advanced calculations, further information types may be obtained, such as, the respiratory pause frequency, RDI (respiratory disturbance index), or the respiration curve, so that it will become easier for the user to determine the necessity of visiting the doctor. Besides, the displaying element can be a digit display (as shown in FIG. 5), LCD, LED or other types of displaying elements, and the power source can be provided by a battery.

In addition, the docking unit 32 can further includes a communication module (not shown) for connecting to an external apparatus in a wired or wireless manner, for example, the wired manner can be achieved by USB, 1394, UART, SPI, Ethernet or other wired transmission interfaces, and the wireless manner can be achieved by one selected from a group consisting of: Bluetooth, IrDa, 802.11x, and other RF communication protocols. Furthermore, for achieving further functions, the docking unit 32 may includes other processing units for dealing with further operations.

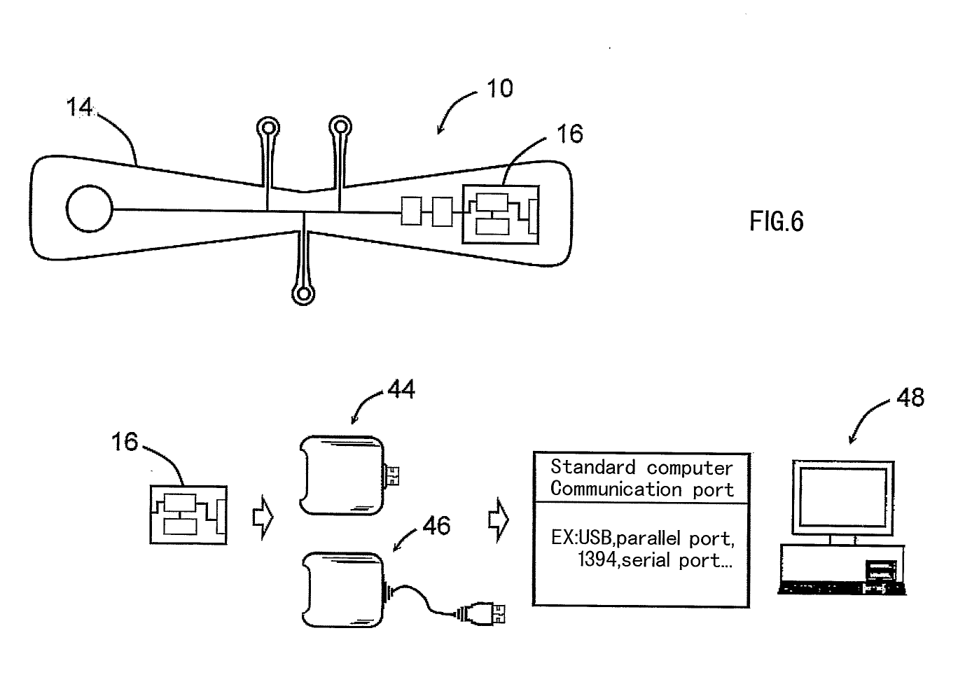

Next, in another embodiment of the present invention, as shown in FIG. 6, the physiological signal sensing device 10 and the computer device 48 are included, and in this situation, some differences from above description are described below.

It is important that the concept of this embodiment is the removable module 16 can be electrically connected to the computer device 48 without any additional circuit arrangement involved, that is, if the user already owns the computer device, there is no need to buy other devices for achieving this connection. Therefore, in this aspect of the present invention, the standard computer communication port, such as, USB, 1394, serial port, and parallel port, plays a key role.

For achieving the destination, a communication interface 28 (as shown in FIG. 3) corresponding to the standard computer communication port is further included in the removable module 16 so that the electrical connection with the computer device 48 can be accomplished. Therefore, there is no need to additionally install special data reading interface on the computer device for downloading the information stored in the removable module 16.

Accordingly, based on the electrical connection between the removable module 16 and the computer device 48 in the present invention, the removable module 16 having the communication interface compatible with the standard computer communication port namely constitutes a conventional computer dongle.

Regarding the electrical connection between the removable module 16 and the computer device 48, except for being implemented as a direct connection, a simple socket adapter 44, 46 can be used, for example, the socket adapter 46 with a connecting cable to the standard computer communication port, or the socket adapter 44 capable of directly connecting to the standard computer communication port are shown in FIG. 6. That is, the communication interface 28 possessed by the removable module 16 is an interface capable of communicating with the standard computer communication port without signal conversion. However, since the connecting structure for the communication interface 28 to the standard computer communication port may occupy a significant space, such as USB connector, there is no need to equip this connecting structure when the sensing device is attached to the body surface, so that it is a preferred way to join the removable module 16 and the socket adapter after the removable module 16 is detached from the carrier 14.

Further, the computer device 48 also can be linked to a remote device and/or remote database through a network so that the medical services, such as online medical database or online medical diagnosis, will not be restricted in geography.

The network can be PSTN (public switched telephone network), Internet or other WANs (wide area networks), and the communication protocol used by the network includes, but not limited, TCP/IP, 802.11x, GSM, PHS and CDMA.

### EXAMPLE I

### The Removable Module Includes the Processor and the Memory

In this example, since the removable module 16 mainly includes (not limited) the processor 22 and the memory 24 besides the communication interface 28, the collected physiological signals and/or the processed physiological information will firstly be stored in the memory 24 and then be downloaded by the computer device 48 through the communication interface 28 after the removable module 16 is detached from the sensing device 10. If needed, further calculation and analysis may be proceeded by the computer device 48.

Alternatively, in addition to the arrangement described above, other modules also can be added if other demands are presented so as to enhance the entire functionality of the sensing device. For example, the RF module 26 can be added to the circuit arrangement for achieving a wireless data transmission, and further, the RF module 26 can also be employed to send out an alarming signal when the physiological signals match a preset condition, such as, lower or higher than a threshold value.

### EXAMPLE II

### The Removable Module Includes the Processor and the RF Module

In this example, since the removable module 16 mainly includes (not limited) the processor 22 and the RF module 26 besides the communication interface 28, the transmission of the collected physiological signals and/or the processed physiological information are wirelessly achieved. Accordingly, the electrical connection between the removable module 16 and the computer device 48 is performed to setup system parameters in the physiological system, such as, ID, sampling rate, resolution, data and time, and so on. Of course, the RF module 26 also can be employed to generate the alarming signal additionally.

Another mode for employing the RF module will be: in a simplest way, the RF module 26 only sends out the alarming signal instead of the general physiological data so that, in this case, the sensing device 10 according to the present invention is equivalent to a physiological alarming and monitoring device.

Furthermore, like Example I, the arrangement of circuits also can be varied for conforming to other practical demands. For example, a memory also can be included in the circuit arrangement for data buffering during the wireless transmission.

Next, in another aspect of the present invention, in addition to collect the physiological signals by the sensing unit 12, a sensor/electrode connecting port can be further provided on the carrier 14 for connecting to an external sensor/electrode, as shown in FIGS. 4A and 4B. In FIG. 4A, the external sensor/electrode is connected to the sensing device 10 through a connector 30, so that the number and type of the sensor/electrode can be varied depending on user's demand, and in FIG. 4B, the external sensor/electrode 31 is directly soldered on the carrier 14.

Here, when the sensing unit on the carrier employs electrode for collecting physiological signals, the type of the external electrode can be identical thereto or different therefrom. That is, for applications, such as regular ECG device and EEG device, all of the electrodes are identical.

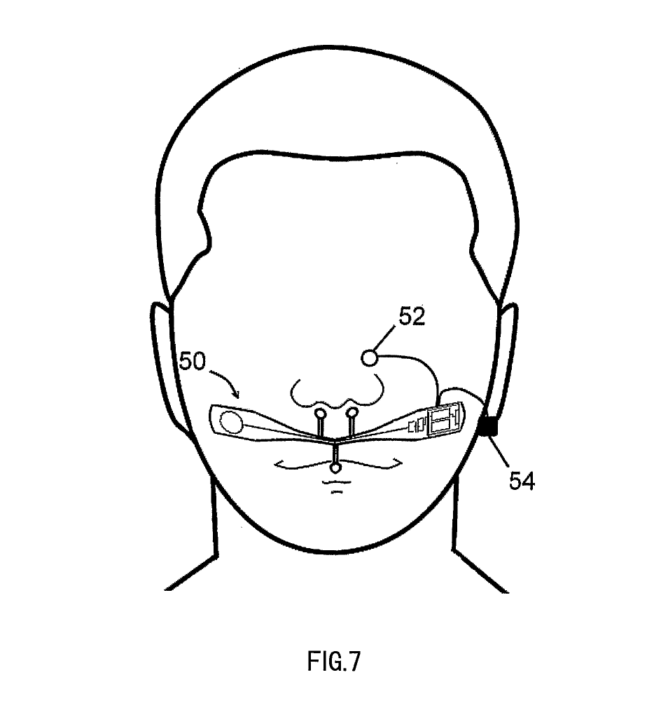

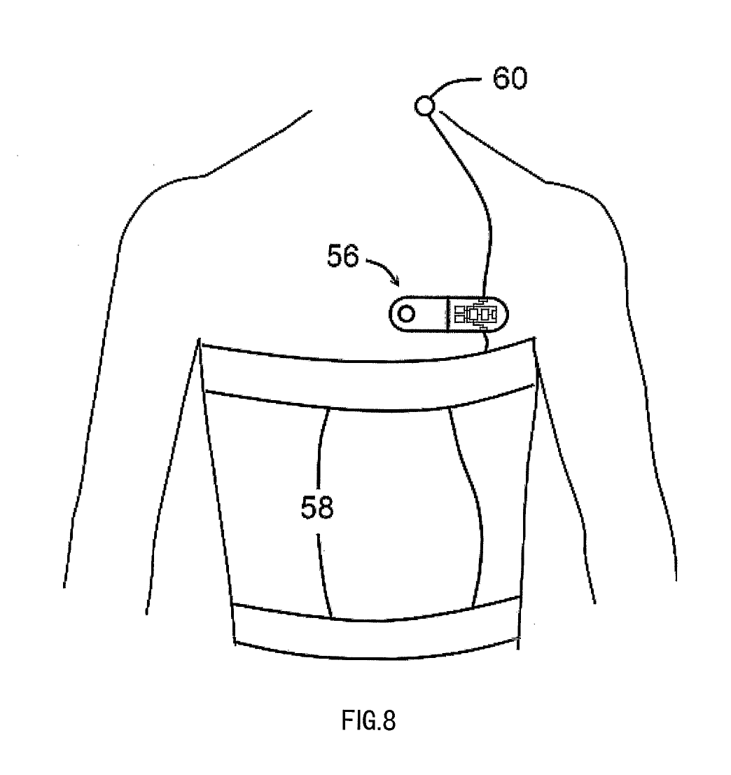

On the other hand, take a respiratory airflow sensing device as an example. The external sensor/electrode connected therewith can be, for example, ECG electrode, EMG electrode, EEG electrode, EOG electrode, SPO2 sensor, and/or snoring sensor, so that according to the present invention, the sensing device can be expanded depending on various demands with minimal add-on cost, and further, the doctor can also obtain more information for better diagnosis. In FIG. 7, a combination is disclosed, wherein the respiratory airflow sensing device 50 is cooperated with a snoring sensor 52 located on the nose and a SPO2 sensor 54 mounted on the ear (which also can be a SPO2 sensor disposed on the forehead). In FIG. 8, another combination includes an ECG device 56, a respiratory effort sensor 58 positioned on the thorax and the abdomen, and a snoring sensor 60 disposed on the throat. Therefore, various combinations are available depending on applications without limitation.

Generally, for portable physiological sensing devices used for long-term and/or multi-signals monitoring, the limitations in weight and size are related to the capacity of memory and power. However, since, owing to the technology development, the density of memory becomes much higher than ever before, it is easier to find a memory with proper capacity and physical size. Therefore, according to the present invention, no matter which design or what kind of combination is performed, it is highly possible to record the details of specific physiological information over a long period, for example, as a respiration detection is implemented, a respiration curve during the whole sleep period can be recorded, that is, not only simply the pause times, but also the retention time and interval for every respiration pause can be recorded, which provides the doctor in-depth information and valuable assistance. Similarly, the same battery used for the portable device also has a longer lasting time due to power-saving development of semi-conductor technology available nowadays. Consequently, even though the present invention employs a simple arrangement, the user still can easily feel the comfort without sacrificing the completeness of data collection.

Exemplarily, when the user wants to know the physiological conditions better, through the sensing device of the present invention, the user can accomplish a basic screening of corresponding physiological data at home easily and conveniently.

On the other hand, if the user wants to monitor multiple parameters or has been informed the categories of parameters to be monitored, the external sensor/electrode can be connected by the electrode/sensor connecting port so as to form the assembled physiological signal sensing device as shown in FIGS. 7 and 8. Therefore, the present invention is also considered as highly scalable.

Next, after measurement, for downloading the physiological information, the user only needs to detach the removable module to connect to the docking unit or the computer device. Alternatively, in the RF module-contained situation, the connection between the removable module and the computer device needs to be established to perform necessary system settings before measurement.

Further preferably, the removable module may act as a computer dongle directly without any additional circuit arrangement involved, so that the present invention is cost effective and operationally convenient.

Besides, through connecting the computer device or the docking unit to a network, the user can transmit the information to the doctor for a preliminary diagnosis or an online diagnosis for saving time, and further, a remote database also can be provided so that, via the network, the user may have a comparison and/or analysis related to the physiological information easily.

In the aforesaid, the present invention provides a physiological signal sensing device which not only has a light weight, but also is able to record multiple physiological signals, and through a removable module, having a communication interface capable of communicating with a standard computer communication port, from the device, the additional reading device in the prior art can be omitted, so that the physiological signal sensing device and the system corresponding thereto in accordance with the present invention do provide an improvement.

The above examples and disclosure are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

1. A physiological signal collecting and monitoring system, comprising:
a physiological signal sensing device, comprising:
an airflow physiological signal sensing unit for detecting airflow physiological signals from a subject;
a circuit arrangement, comprising a processor and a memory, wherein the processor executes a preloaded program to receive the physiological signals from the physiological signal sensing unit, process thereof and store resultant physiological information into the memory; and
a flexible carrier for carrying the airflow physiological signal sensing unit and the circuit arrangement to detect the airflow physiological signal at the nostrils and the mouth of the subject; and
a computer device, having a standard computer communication port, for further processing the physiological information,
wherein
the sensing device further comprises a communication interface to communicate with the standard computer communication port, and the communication interface together with the processor and the memory are integrated into a removable module; and
the removable module is detachable from the sensing device for acting as a computer dongle so that the removable module is capable of electrically connecting with the computer device to download the physiological information stored in the removable module into the computer device.

2. A physiological signal collecting and monitoring system as claimed in claim 1, wherein the airflow physiological signal sensing unit is an airflow pressure sensor or a thermal sensor.

3. A physiological signal collecting and monitoring system as claimed in claim 2, wherein the physiological signal sensing device further comprises a sensor/electrode connecting port for connecting with an external sensor/electrode, and said external sensor/electrode is selected from a group consisting of a respiratory sensor, a blood oxygenation sensor, a snoring sensor, an ECG electrode, an EMG electrode, an EOG electrode and an EEG electrode.

4. A physiological signal collecting and monitoring system as claimed in claim 1, wherein the removable module is separated from the physiological signal sensing device by setting a connector therebetween, tearing, cutting or other separating methods.

5. A physiological signal collecting and monitoring system as claimed in claim 1, wherein the circuit arrangement further comprises a signal processing circuit having functions of filtering and amplifying for processing the detected physiological signals, and/or the circuit arrangement further comprises an A/D (analog-to-digital) converter for converting the physiological signals into a digital form.

6. A physiological signal collecting and monitoring system as claimed in claim 1, wherein the circuit arrangement further comprises a RF module for executing a wireless input and output.

7. A physiological signal collecting and monitoring system as claimed in claim 6, wherein the RF module is implemented to wirelessly output an alarm signal when the physiological signals match a preset condition.

8. A physiological signal collecting and monitoring system as claimed in claim 6, wherein the RF module is integrated into the removable module.

9. (canceled)

10. A physiological signal collecting and monitoring system as claimed in claim 1, wherein the electrical connection between the removable module and the computer device is achieved by a socket adapter.

11. A physiological signal collecting and monitoring system as claimed in claim 1, wherein the computer device is a personal computer, a notebook computer, a PDA (personal digital assistant), or other computing devices having the standard computer communication port, and the standard computer communication port is selected from a group consisting of: USB, 1394, serial port, parallel port and other wired transmission interface.

12.-19. (canceled)
